(19) United States
(12) Patent Application Publication
(10) Pub. No.: US 2020/0129975 A1
(43) Pub. Date: Apr. 30, 2020

(54) PARYLENE-A-COATED INSOLUBLE POROUS MEMBRANE-BASED PORTABLE UREA BIOSENSOR FOR USE IN FLOW CONDITIONS

(71) Applicant: INDUSTRY ACADEMIC COOPERATION FOUNDATION, HALLYM UNIVERSITY, Chuncheon-si (KR)

(72) Inventors: Min PARK, Chuncheon-si (KR); Gun Yong SUNG, Seoul (KR); Jee Young KIM, Seoul (KR); Kyunghee KIM, Chuncheon-si (KR)

(73) Assignee: INDUSTRY ACADEMIC COOPERATION FOUNDATION, HALLYM UNIVERSITY, Chuncheon-si (KR)

(21) Appl. No.: 16/666,696

(22) Filed: Oct. 29, 2019

(30) Foreign Application Priority Data
Oct. 29, 2018 (KR) 10-2018-0129590

(51) Int. Cl. B01L 3/00; C12Q 1/00

(52) U.S. Cl. CPC B01L 3/502; C12Q 1/002; B01L 2300/12; B01L 2300/0645; B01L 2300/16

(57) ABSTRACT

Provided is a portable urea sensor which can be used under a flow condition by using a porous polytetrafluoroethylene (PTFE) membrane coated with parylene-A, which is parylene functionalized with an amine by vacuum deposition. To produce a specific electrochemical sensor signal from urea, urease, which is an enzyme hydrolyzing urea, is immobilized to a parylene-A-coated PTFE membrane by chemical crosslinking using glutaraldehyde. The urea-immobilized membranes are assembled in a polydimethylsiloxane (PDMS) fluid chamber, and a screen-printed carbon 3-electrode system is used. The success of the urease immobilization process is confirmed using scanning electronmicroscopy (SEM) and Fourier-transform infrared (FTIR) spectroscopy. The optimal concentration of urease to be immobilized to the parylene-A-coated PTFE membrane is determined to be 48 mg/mL, and the optimal number of the membranes in the PDMS chamber is determined to be 8.

### CROSS-REFERENCE TO RELATED APPLICATION

This application claims priority to and the benefit of Korean Patent Application No. 10-2018-0129590, filed on Oct. 29, 2018, the disclosure of which is incorporated herein by reference in its entirety.

### BACKGROUND

### 1. Field of the Invention

The present invention relates to a parylene-A-coated insoluble porous membrane-based portable urea biosensor to be used under a flow condition.

Abbreviations used in the present invention are as follows:

PTFE: Polytetrafluoroethylene; PDMS: Polydimethylsiloxane,

PBS: Phosphate buffered saline; SEM: Scanning electron microscopy;

FTIR: Fourier-transform infrared; FITC: Fluorescein isothiocyanate;

AP: Parylene-A-coated PTFE; UAP: Urease-immobilized AP; and

LOD: Limit of detection; ESRF: End-stage renal failure.

### 2. Discussion of Related Art

Urea is a compound synthesized from ammonia during the breakdown of proteins in the liver, and the final nitrogen product of metabolism. Ammonia, which is highly toxic in mammals and amphibians, is transformed into urea which is relatively less toxic by the ornithine cycle. The synthesized urea is stored in the kidneys and excreted from the body via urine. Urea is widely used with creatine as a significant indicator for kidney function. Proper ranges of urea and creatinine in the blood are 7 to 20 mg/dL and 0.7 to 1.2 mg/dL, respectively [1-3]. The kidney is a bean-shaped secretory organ with many functions such as the excretion of biological waste, the maintenance of acid, base and electrolyte metabolisms, blood maintenance, and the production and activation of parathyroid hormones regulating calcium and phosphate metabolisms. The kidney is essential for the living body, and 20 to 25% of the blood flowing out from the heart goes into the kidneys. The total blood volume filtered by the kidneys is 180 L daily. Most of the water filtered from the blood is reabsorbed, and thus only 1 to 2 L is excreted as urine. A condition in which kidney function is impaired such that homeostasis cannot be maintained is called renal failure. In renal failure, urea and creatinine concentrations in serum increase due to a sharp fall in the glomerular filtration rate. Acute renal failure may occur after complicated surgery due to a severe injury or lack of blood supply to the kidneys. In this case, kidney function may be restored to normal after treatment. Meanwhile, in acute renal failure, there are no specific symptoms in its early stage, and various symptoms such as hypertension and diabetes will be observed later. When chronic renal failure persists and worsens, it may progress to end-stage renal failure (ESRF). For end-stage renal failure, kidney transplantation is essential, and hemodialysis or peritoneal dialysis is needed during the transplantation [14, 15]. Urea and creatinine concentrations in the blood and peritoneum are the major indicators of dialysis progression.

To monitor a urea concentration, various analysis methods and biosensors based on electrochemical, thermal, optical and piezoelectric detection were developed [16-20]. Among these, electrochemical urea biosensors have been widely developed due to high sensitivity and possibility of efficient and rapid analysis [21]. For electrochemical measurement of a urea concentration, a urease-based biosensor was developed [22]. Ureases are nickel-containing metalloenzymes, which are found in numerous bacteria, fungi, algae and plants [23]. Urease catalyzes the hydrolysis of urea into carbon dioxide and ammonia. The hydrolysis of urea is as follows:

(NH₂)₂CO (urea)+H₂O→CO₂+2NH₃

Urease hydrolyzes urea into ammonia and carbamate molecules. Unstable carbamate is then degraded into a second ammonia molecule and a carbon dioxide molecule. In an aqueous solution, ammonia is present as charged ammonium ions, and an electrochemical signal is generated in the hydrolysis of urea by urease. The intensity of the signal is directly proportional to the amount of ammonium ions present in the solution, and thus a urease-based electrochemical biosensor facilitates qualitative analysis. To achieve high sensitivity, a high concentration of urease that can produce a detectable amount of ammonium ions is needed. Therefore, it is necessary to immobilize a high density of urease. In previous research, urease was immobilized on the surface of an electrode used in electrochemical measurement. However, according to direct immobilization, a urease-immobilizable region is limited to an electrode region, and since urease should be immobilized again every time the electrode is replaced, analysis costs increase. As a better alternative, a nano structure to which urease can be immobilized is constructed on the surface of the electrode. However, this type of biosensor requires the reimmobilization of urease after the replacement of an electrode [21, 24, 25]. In a recent paper, urease was immobilized to a separate substrate tightly immobilized to an electrode, the concentration of urea was electrochemically measured, and the urease was immobilized to a porous substrate to increase an immobilizing area and enhance sensitivity [26]. However, in these studies, urea measurement was performed under a static condition [22]. The measurement of a urea concentration in a static state after blood and urine sampling is useful in medical diagnosis. However, to monitor the progression of dialysis during blood or peritoneal dialysis, a urea concentration should be measured while it flows. The use of a biological reactor for flow analysis was reported recently [27]. However, since this biological reactor needs a long reaction time through a complicated reaction process, and measures only one optical signal from the single injection of urea, it is considered that this biological reactor is not suitable for continuously monitoring urea in physiological samples. Ohnishi et al. measured a urea concentration using a microfluidic chip [28]. This device used a flow channel, but since a charge was measured under a static condition, it was able to be used for only a single measurement. In addition, a thermal biosensor was used in flow injection analysis [29]. However, a detected urea concentration (100 mM) was much higher than the normal range, and continuous monitoring was impossible. In any case, to monitor urea in physiological samples in real time, a biosensor should be developed to continuously monitor urea while it flows.

### SUMMARY OF THE INVENTION

The present invention is directed to providing a urea sensor which may be sensitive to and rapidly detect urea under a flow condition.

In addition, the present invention is directed to providing a urea sensor which is portable and convenient to use.

In addition, the present invention is directed to providing a urea sensor which is not decreased in sensitivity even by repeated use.

To attain the above-mentioned objects, a real-time urea monitoring biosensor using a membrane was manufactured to immobilize a high concentration of urease to a porous membrane and used under a flow condition. To immobilize urease at a high density, the porous membrane was coated with parylene-A, which is an amine-functionalized parylene. Parylene (poly(p-xylylene)) is a polymer which may be applied on a porous membrane by vapor deposition at room temperature. Parylene-A contains one amine group per repeat unit. Therefore, a porous membrane was first coated with parylene-A, thereby forming a high concentration of amino groups thereon, and a high concentration of urease was immobilized on the membrane using glutaraldehyde as a crosslinking agent [30]. The porous membrane was increased in sensitivity of electrochemical measurement by maximizing a contact area with a fluid under a flow condition. Finally, the urease-immobilized porous membrane was inserted into a polydimethylsiloxane (PDMS) chamber, thereby forming a fluid system. And then, a urea concentration was monitored in a flow condition using the manufactured urea biosensor.

In one aspect, the present invention provides a portable urea biosensor, which includes:

a fluid chamber consisting of a material that does not block an electrical signal;

parylene-A-coated insoluble porous membranes which are placed in the fluid chamber, and to which urease is immobilized by a chemical bond;

a screen-printed 3-electrode system, which is adjacent to the urease-immobilized parylene-A-coated insoluble porous membrane to which the urease is immobilized by a chemical bond and includes a working electrode, a counter electrode and a reference electrode sensing an electrochemical signal generated from the membrane;

a sample inlet through which a flowing sample is introduced into the fluid chamber; and

a sample outlet through which the sample flows out from the fluid chamber, wherein the portable urea biosensor measures a urea concentration by an electrochemical method with respect to a flowing sample.

In addition, in the present invention, the urease is immobilized to the parylene-A-coated insoluble porous membrane by chemical crosslinking using glutaraldehyde.

In addition, in the present invention, the insoluble porous membrane is manufactured of one or more biocompatible materials selected from the group consisting of fucoidan, collagen, alginate, chitosan, hyaluronic acid, silk fibroin, a polyimide, polyamix acid, polycarprolactone, polyetherimide, nylon, polyaramid, polyvinyl alcohol, polyvinylpyrrolidone, poly-benzyl-glutamate, polyphenyleneterephthalamide, polyaniline, polyacrylonitrile, polyethylene oxide, polystyrene, cellulose, polyacrylate, polymethylmethacrylate, polylactic acid (PLA), polyglycolic acid (PGA), a copolymer (PLGA) of PLA and PGA, poly{poly(ethylene oxide)terephthalate-co-butyleneterephthalate} (PEOT/PB T), polyphosphoester (PPE), polyphosphazene (PPA), polyanhydride (PA), polytetrafluoroethylene (PTFE), poly(ortho ester) (POE), poly(propylene fumarate)-diacrylate (PPF-DA) and poly(ethylene glycol) diacrylate (PEG-DA). In an exemplary embodiment of the present invention, an insoluble porous membrane manufactured of polytetrafluoroethylene (PTFE) was used. However, as long as a porous membrane is insoluble in water or an aqueous solution and has numerous pores, even though not being formed of PTFE, the present invention is not limited thereto. The above-mentioned materials are merely exemplary, but the present invention is not limited to membranes formed of these materials.

In addition, the present invention further includes housings surrounding the fluid chamber and the 3-electrode system.

In addition, in the present invention, the working electrode of the 3-electrode system is aminated.

In another aspect, the present invention provides a urea biosensor in which the 3-electrode system is connected to an external electrochemical analyzer to detect an electrochemical signal in a fluid chamber.

In still another aspect, the present invention provides an insoluble porous membrane for immobilizing a protein coated with an amine-functionalized parylene film. Various proteins such as enzymes including urease may be immobilized to this membrane, and thus the membrane can be used as a sensor.

In addition, in the present invention, the insoluble porous membrane is manufactured of one or more biocompatible materials selected from the group consisting of fucoidan, collagen, alginate, chitosan, hyaluronic acid, silk fibroin, a polyimide, polyamix acid, polycarprolactone, polyetherimide, nylon, polyaramid, polyvinyl alcohol, polyvinylpyrrolidone, poly-benzyl-glutamate, polyphenyleneterephthalamide, polyaniline, polyacrylonitrile, polyethylene oxide, polystyrene, cellulose, polyacrylate, polymethylmethacrylate, polylactic acid (PLA), polyglycolic acid (PGA), a copolymer (PLGA) of PLA and PGA, poly{poly(ethylene oxide)terephthalate-co-butyleneterephthalate} (PEOT/PB T), polyphosphoester (PPE), polyphosphazene (PPA), polyanhydride (PA), polytetrafluoroethylene (PTFE), poly(ortho ester) (POE), poly(propylene fumarate)-diacrylate (PPF-DA) and poly(ethylene glycol) diacrylate (PEG-DA). In one exemplary embodiment of the present invention, an insoluble porous membrane manufactured of PTFE was used. However, as long as a porous membrane is insoluble in water or an aqueous solution and has numerous pores, even though not being formed of PTFE, the present invention is not limited thereto. The above-mentioned materials are merely exemplary, but the present invention is not limited to membranes formed of these materials.

In yet another aspect, the present invention provides a method of manufacturing an insoluble porous membrane for immobilizing a protein, which includes:

(1) uniformly depositing an amine-functionalized parylene film on a porous membrane at room temperature; and

(2) after deposition of the parylene film, converting an amine group on the surface of the parylene film into an active aldehyde group by a reaction with a glutaraldehyde solution as a crosslinking agent.

In addition, in the present invention, the step (1) is performed in a vacuum state.

In yet another aspect, the present invention provides a method of manufacturing a urease-immobilized insoluble porous membrane, which includes:

(1) uniformly depositing an amine-functionalized parylene film on an insoluble porous membrane at room temperature;

(2) after deposition of the parylene film, converting an amine group on the surface of the parylene film into an active aldehyde group by a reaction with a glutaraldehyde solution as a crosslinking agent; and

(3) immobilizing urease to the insoluble porous membrane by a chemical reaction of the active aldehyde group on the surface of the parylene film and urease having a free amine group.

In yet another aspect, the present invention provides a urease-immobilized insoluble porous membrane for a urea biosensor, which is manufactured by the above-described method, and reduces noise in the measurement of a urea concentration due to the decrease in non-specific reactions.

In yet another aspect, the present invention relates to a method of measuring a urea concentration in a flowing sample by an electrochemical method using a portable urea biosensor, which includes a fluid chamber; parylene-A-coated insoluble porous membranes which are placed in the fluid chamber, and to which urease is immobilized by a chemical bond; a screen-printed 3-electrode system, which is adjacent to the urease-immobilized parylene-A-coated insoluble porous membrane to which the urease is immobilized by a chemical bond, and includes a working electrode, a counter electrode and a reference electrode sensing an electrochemical signal generated from the membrane; a sample inlet through which a flowing sample is introduced into the fluid chamber; and a sample outlet through which the sample flows out from the fluid chamber.

In addition, in the present invention, the flowing sample flows at a rate of 0.5 to 10 mL/min.

In addition, in the present invention, the number of the urease-immobilized parylene-A-coated insoluble porous membranes is 6 to 10.

In addition, in the present invention, a urea concentration in the sample ranges from 0.6 to 20 mM.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The above and other objects, features and advantages of the present invention will become more apparent to those of ordinary skill in the art by describing in detail exemplary embodiments thereof with reference to the accompanying drawings, in which:

FIGS. 1A and 1B show a set of images of (a) the configuration of a sensor system and (b) a sensor unit;

FIGS. 2A-2D show a set of fluorescence microscope images of a fluorescein isothiocyanate (FITC)-treated parylene-N-coated PTFE membrane, a FITC-treated PTFE membrane, a FITC-treated parylene-A-coated PTFE membrane and a fluorescein-treated parylene-A-coated PTFE membrane;

FIG. 3 shows a set of SEM microscope images of a PTFE membrane, a parylene-A-coated PTFE membrane, a glutaraldehyde-treated parylene-A-coated PTFE membrane and a urease-immobilized parylene-A-coated PTFE film;

FIG. 4 shows the FTIR spectra of a PTFE membrane, a parylene-A-coated PTFE membrane, a glutaraldehyde-treated parylene-A-coated PTFE membrane, and a urease-immobilized parylene-A-coated PTFE membrane;

FIG. 5 is a graph measuring urease activity and optimizing a urease concentration with an ammeter;

FIG. 6 shows a result of detecting urea with various numbers of UAP membranes; and

FIGS. 7A-7C show a result of real-time monitoring of urea using a UAP membrane-based biosensor in PBS at various flow rates, a peritoneal dialysate, and a urea sensor reaction.

### DETAILED DESCRIPTION OF EXEMPLARY EMBODIMENTS

Hereinafter, the configuration of the present invention will be described in further detail with reference to specific embodiments. However, it is apparent to those of ordinary skill in the art that the scope of the present invention is not limited only to the description of the embodiments.

Reagents

Urease (derived from Canavalia ensiformis), urea, glutaraldehyde, disodium hydrogen phosphate, potassium dihydrogen phosphate, fluorescein, fluorescein isothiocyanate (FITC) and ammonium carbamate were purchased from Merck (Darmstadt, Germany). A urea analysis kit was purchased from BioAssay Systems (CA, US). Phosphate buffered saline (PBS) was purchased from LPS Solution (Daejeon, Korea), and used as a supporting electrolyte for electrochemical measurement. The PBS solution was prepared by mixing 20 mM disodium hydrogen phosphate and 20 mM potassium dihydrogen phosphate (pH=7.4), and used as an immobilization buffer solution. A porous polytetrafluoroethylene (PTFE) membrane having a pore size of 1 μm was purchased from Advantec MFS (CA, US). Lung and peritoneal dialysates of chronic renal failure patients were obtained from Seoul National University Hospital in accordance with the Declaration of Helsinki. This study was approved by the Institutional Review Board of Seoul National University Hospital and Hallym University.

Parylene Coating on PTFE Membrane and Urease Immobilization

As is typical, a porous PTFE membrane was punched with a biopsy punch (diameter: 8 mm), and a parylene film was deposited according to the following procedure. (1) An amine-functionalized parylene dimer was evaporated at 160 □. (2) The dimer was pyrolyzed at 650 □ to produce an amine-functionalized p-xylene radical, which is highly reactive. (3) The amine-functionalized parylene film was uniformly deposited on the PTFE film at room temperature. During the entire coating process, a vacuum condition (<5 Pa) was maintained. After deposition of the parylene film, the amine group on the surface of the film converted into an active aldehyde group by vigorous agitation of a 10% glutaraldehyde solution as a crosslinking agent dissolved in a PBS solution for one hour. Finally, the urease was immobilized to the PTFE membrane by a chemical reaction between an active aldehyde group and a free amine group of the urease.

Measurement of Urease Activity

A commercial urea assay kit was used to measure a urea concentration. For example, the urease-immobilized PTFE membrane was reacted with 16.67 mM urea at 25 □ for 1 hour while stirring. After the hydrolysis of urea by urease on the urease-immobilized PTFE membrane, 5 μL of the hydrolyzed urea solution was transferred into a 96-well plate, and then 200 μL of a phthalaldehyde reagent was added to allow a reaction for 20 minutes. Subsequently, the activity of the immobilized urease was measured at a wavelength of 520 nm through measurement of optical density according to a colorimetric method.

Manufacture of Urea Biosensor

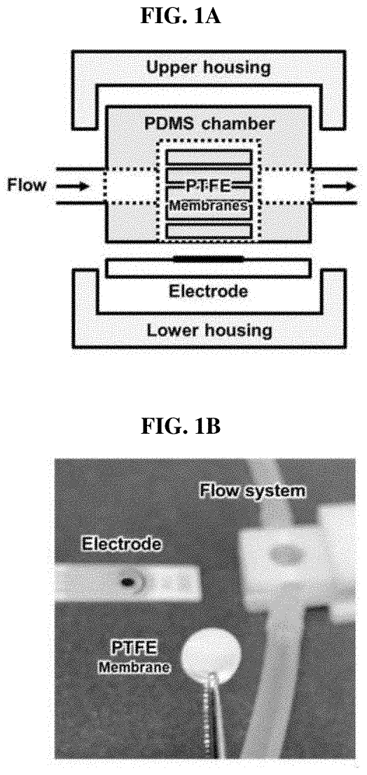

The urea biosensor manufactured in the present invention is illustrated in FIGS. 1A and 1B. The biosensor consists of a lower housing, a 3-electrode system, a polydimethylsiloxane (PDMS) fluid chamber, urease-immobilized PTFE membranes (4, 6, 8, 10 or 12) and an upper housing. The housings were 3D-printed using an acrylonitrile polybutadiene styrene (ABS) filament. To manufacture the PDMS fluid chamber, PDMS was solidified on a Si wafer attached to a plastic frame (width=10.20 mm, length=14 mm, height=5.43 mm). After solidification, a cylindrical fluid chamber having an inner diameter of 8 mm was punched using a biopsy punch. The screen-printed 3-electrode system included a working electrode with a diameter of 4 mm (DropSens, Llanera, Spain), and was used after amination [26]. For amination, the electrode was immersed in a 0.5M ammonium carbamate solution, and subjected to cyclic voltammetry (CV). Sweep potential and speed were set to 0.5 to 1.2V and 50 mV/s during 50 cycles, respectively. After amination, the electrode was washed with purified water. After preparation of all sections of the urea biosensor, the urease-immobilized parylene-coated PTFE membrane was put into a PDMS fluid chamber, and as shown in FIG. 1A, other parts were assembled.

Configuration of Flow System and Electrochemical Measurement

As shown in FIG. 1B, to detect a urea concentration under a flow condition, a single flow system was configured. A flow cell of the urea sensor was linked to a tube, and an open circuit was formed by the flow. A flow rate was adjusted from the open circuit linked to an inlet tube using a peristaltic pump (Ismatec, Wertheim, Germany), and an outlet tube was linked to a bottle. Potentiostats (DropSens, Llanera, Spain) were connected to the electrode installed in the sensor to measure a current generated by the hydrolysis of urea by urease. For real-time monitoring of urea under a flow condition, chronoamperometry was performed at 1.1 V with various flow rates.

Result 1: Manufacture of Urease-Immobilized PTFE Membrane Using Parylene-A Coating

Electrochemical reactions occur at an electrode surface. Therefore, in an enzyme-based electrochemical biosensor, the enzyme is generally immobilized on the electrode surface. In such type of biosensor, since the enzyme and the electrode have to be replaced at the same time when the enzyme activity decreases due to repeated measurements, the lifespan of the electrode is shortened, and the cost of the biosensor increases. In addition, the enzyme immobilization region of the biosensor is restricted to the area of the electrode. In addition, the immobilization environment is determined by the electrode material, which makes the immobilization of the enzyme highly specific.

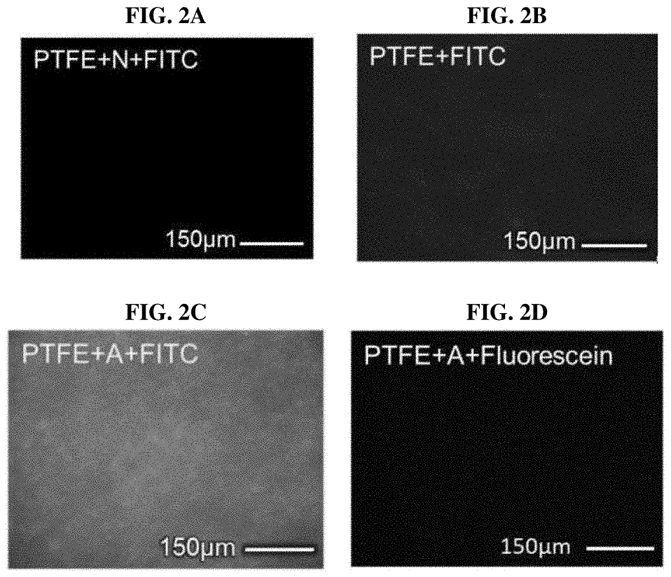

In the present invention, first, urea was immobilized to a porous membrane having a large surface area, and then a sensor based on a urease-immobilized membrane was manufactured to monitor urea under a flow condition. To immobilize the urease, a PTFE membrane having excellent drug resistance was selected. To form a urease-immobilized region, the membrane was coated with parylene-A by vapor deposition. The parylene has excellent chemical resistance and excellent mechanical properties. Chloride group-containing parylene C has been approved by the Food and Drug Administration (FDA). In the present invention, parylene-A was uniformly applied on the surface of the porous PTFE membrane at room temperature by vapor deposition. The parylene-A has one amine group per repeat unit. Therefore, as the parylene-A is deposited on the PTFE membrane, the membrane surface may be modified with an amine group. Subsequently, the membrane was treated with glutaraldehyde, which is a dialdehyde crosslinking agent, thereby functionalizing the surface with an aldehyde group through the reaction between an amine and an aldehyde. Afterward, urease was immobilized on the membrane through the reaction between the amine group of the enzyme and the aldehyde group on the membrane surface. It is considered that the chemical crosslinking of the urease on the membrane is more stable than physical adsorption under a flow condition. Therefore, such enzyme immobilization strategy prevents the biodegradation of the urease and improves the durability of the urea sensor, and thus the biosensor can be used for a long time. The amination by parylene-A coating was analyzed using a fluorescence microscope (FIGS. 2A-2D). Here, a 100-nm-thick coating was implemented on the porous PTFE membrane using 100 mg of a parylene dimer. The amine group was visualized by FITC through a specific reaction between an amine and an isothiocyanate group. As shown in FIG. 2A, when a unfunctionalized parylene-N-coated PTFE membrane was treated with 1 μg/mL of FITC, fluorescence was not observed. However, weak fluorescence was observed from the FITC-treated porous PTFE membrane (FIG. 2B), which may be caused by non-specific binding between FITC and PTFE. Since the PTFE membrane is hydrophilic, FITC non-specifically binds thereto. However, parylene, which is a hydrophobic polymer, blocks non-specific binding. Therefore, when the parylene-A-coated PTFE (AP) membrane is treated with FITC, strong and uniform fluorescence is observed (FIG. 2C). In contrast, when unfunctionalized fluorescein is applied to the AP membrane, a fluorescent signal was not detected (FIG. 2D). Therefore, comparing FIG. 2B with FIGS. 2A and 2D, it was confirmed that parylene coating prevents non-specific binding. This shows that parylene coating prevents various biomolecules injected in a flowing state from binding to a urease-immobilized AP (UAP) membrane, thereby reducing the noise of the biosensor, that is, unnecessary information. In addition, the comparison of FIG. 2C with FIGS. 2A and 2D shows that only the amine-containing parylene layer and ITC-conjugated fluorescein react with each other, and there is no reaction between parylene-N and FITC or parylene-A and fluorescein. This indicates that ITC is an amine-specific binding group, and parylene-A-coating makes the surface of the PTFE membrane uniformly functionalized with a high-density active amine group, and prevents non-specific binding.

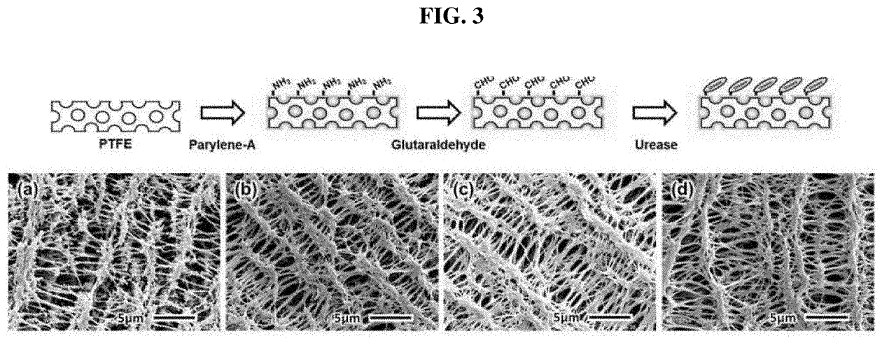

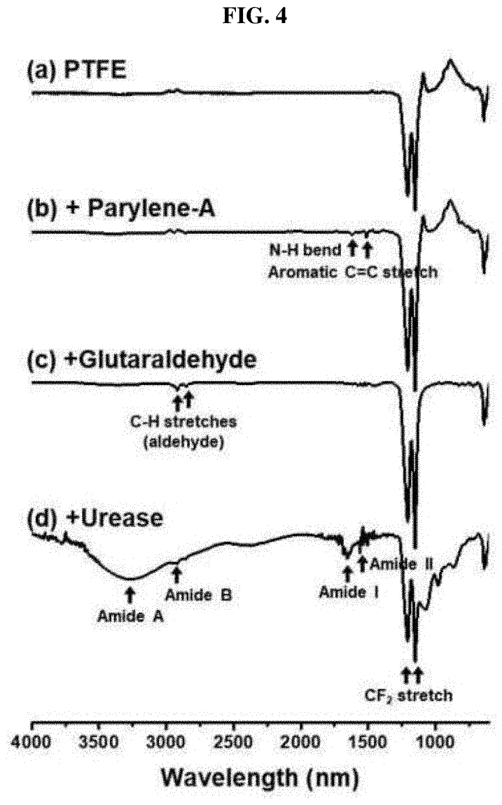

A urease-immobilized PTFE membrane was observed using a 5,000× scanning electron microscope (SEM). After deposition of parylene-A, the microporous structure of the PTFE membrane ((a) of FIG. 3) was maintained ((b) of FIG. 3). In addition, after glutaraldehyde treatment ((c) of FIG. 3) and urease immobilization ((d) of FIG. 3), the microstructure of the PTFE membrane was not changed. Therefore, the deposition of parylene-A on the PTFE membrane did not affect the microporous structure of the membrane. In addition, as a result of observation by a SEM, it was confirmed that a uniform parylene-A layer was formed on the PTFE membrane. Furthermore, chemical treatment performed with a crosslinking agent and urease did not affect the microporous structure of the PTFE membrane. Based on this result, it was confirmed that a UAP membrane having an undamaged microporous structure was manufactured. Since the UAP membrane needs to be stacked in a PDMS fluid chamber, the maintenance of the porous structure is essential for the flow of a urea sample. The change in functional group on the membrane surface during urease immobilization was analyzed using Fourier-Transform Infrared Spectroscopy (FTIR). As shown in (a) of FIG. 4, in the spectra of the PTFE membrane, two CF2 stretching peaks were shown at 1205 cm⁻¹ and 1150 cm⁻¹. In the spectra recorded after the membrane was coated with parylene-A, additional peaks corresponding to a N—H band and aromatic C═C stretching were observed at 1622 cm⁻¹ and 1513 cm⁻¹, respectively ((b) of FIG. 4). Accordingly, it was confirmed that the PTFE membrane was coated with parylene-A having an amine group on the phenyl group-containing backbone (p-xylene). When the AP membrane was treated with glutaraldehyde, an amine peak disappeared, and new C—H stretching peaks corresponding to an aldehyde group are shown at 2919 cm⁻¹ and 2850 cm⁻¹ ((c) of FIG. 4), indicating that the parylene-A-coated amine group has reacted with the aldehyde group of the glutaraldehyde, and the surface of the parylene-A-coated PTFE membrane is functionalized with an aldehyde group. The thickness of the parylene-A layer is 100 nm, which is much smaller than the IR penetration depth, which is several μm. Therefore, the CF2 stretching peak is much stronger than the amine or aldehyde peak. After urease immobilization, additional peaks generated by amide A, amide B, amide I and amide II were observed at 3270, 2925, 1652 and 1559 cm⁻¹, respectively ((d) of FIG. 4). Such amide peaks are typical peaks for proteins, confirming that urease is immobilized to the AP membrane. From this data, it was confirmed that urease is immobilized via the following steps: (1) The surface of a porous PTFE membrane was modified with an active amine group by the deposition of parylene-A. (2) Subsequently, the surface of the AP membrane was modified with an aldehyde group by the treatment of glutaraldehyde. (3) A free amine group-containing urease was immobilized to the AP membrane by chemical crosslinking. The urea biosensor of the present invention may reinforce sensitivity and reduce noise using the UAP membrane manufactured thereby. The sensitivity improvement is caused by maximizing an enzyme immobilization region due to the porous structure of the membrane, and the noise improvement is caused by the blockage of non-specific binding by parylene.

Result 2: Optimization of Urease-Immobilized PTFE Membrane-Based Urea Sensor

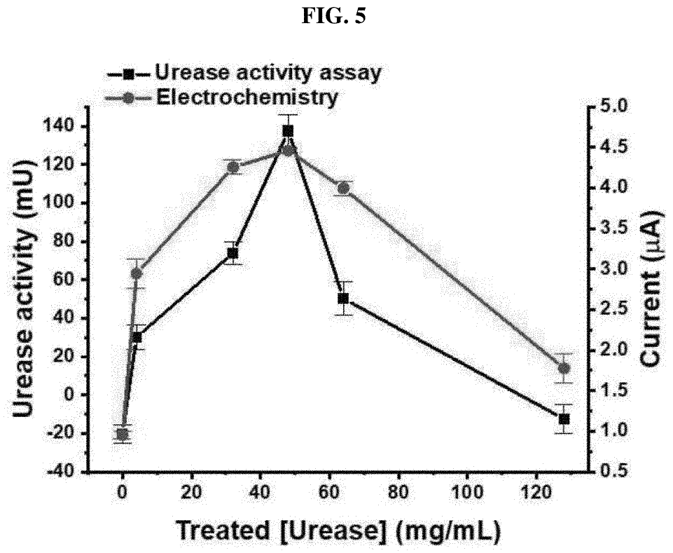

The urea biosensor manufactured based on the UAP membrane is illustrated in FIG. 1A. The UAP membrane was placed in a PDMS fluid chamber. An electrode system was put on the bottom of the chamber while in contact with the UAP membrane. A urea sample was injected into the PDMS chamber, and the sample was flowed through the PDMS chamber via pores of the UAP membrane. Subsequently, the urea present in the sample was hydrolyzed by urease immobilized on an AP membrane, thereby producing a sensor signal. The produced signal was measured using a screen-printed 3-electrode system including a carbon working electrode, a counter electrode and a reference electrode. Before use, to improve sensitivity and stability, the working electrode was treated with carbamic acid for amination. To test urea immobilization efficiency and optimize a urease concentration in immobilization, 0, 4, 32, 48, 64 and 128 mg/mL of urease were used. After immobilization, the activity of immobilized urease was tested using a urease activity assay kit. A standard urea sample (16.67 mM) was added to the UAP membrane, and maintained at 25 □ for 1 hour, followed by measurement of the concentration of remaining urea. The activity of the immobilized urease was calculated according to an amount of hydrolyzed urea. One U of urease liberates 1.0 μmol of NH₃ per minute at pH 7.0 and 25 □. As the concentration of the urease used in immobilization increased, the activity of immobilized urease (▪) increased (FIG. 5), and when 48 mg/mL of urease was used, the maximum activity was 137.5 mU. However, at higher concentrations, the activity of the immobilized urease decreased. This shows that 48 mg/mL has the highest amount of urease is immobilized to the AP membrane by glutaraldehyde crosslinking. The use of a higher concentration of urease leads to the decrease in urea hydrolysis epitopes or the Hook effect, which makes the activity of the UAP membrane lowered. The optimization of the urease concentration was tested by electrochemical measurement. The UAP membranes which were previously treated with various concentrations of urease were inserted into a PDMS chamber, and 10 mM of stationary urea was monitored using an ammeter at an applied voltage of 1.1V. As shown in FIG. 5, as the concentration of the urease used in treatment increased, a current (●) increased, and the maximum value of 4.46 μA was measured on the UAP membrane obtained by treatment with 48 mg/mL urease. At higher concentrations, a current decreased. The data perfectly corresponded with the urease activity assay result. This shows that the sensitivity of the urea biosensor is increased with the increase in the activity of the urease immobilized onto the AP membrane. The optimal concentration for urease immobilization was determined to be 48 mg/mL through urease activity assay and electrochemical measurement. To increase the durability and surface area of the porous PTFE membrane, the membrane was coated with a parylene-A layer for urease immobilization. The optimal concentration, that is, 48 mg/mL of urease, was immobilized to the AP membrane using glutaraldehyde, which is a crosslinking agent, and a urea biosensor which real-time monitors urea under a flow condition was manufactured using the UAP membrane optimized as described above.

Result 3: Real-Time Monitoring of Urea Concentration Under Flow Condition

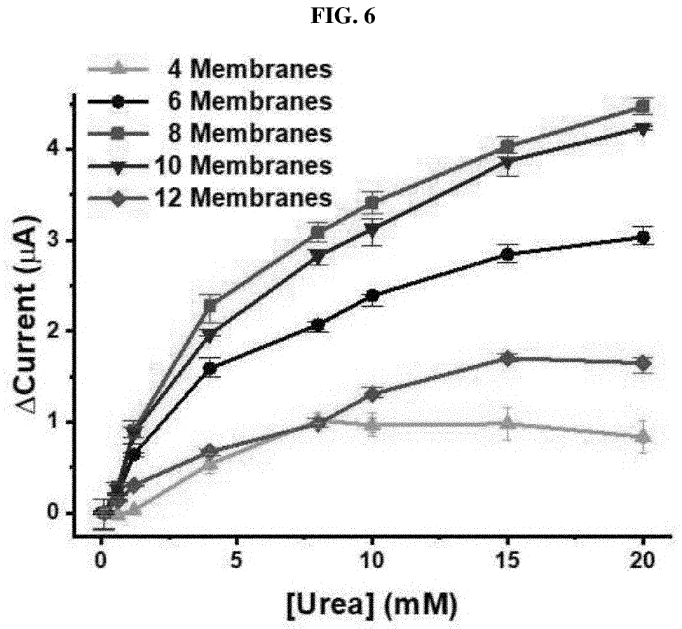

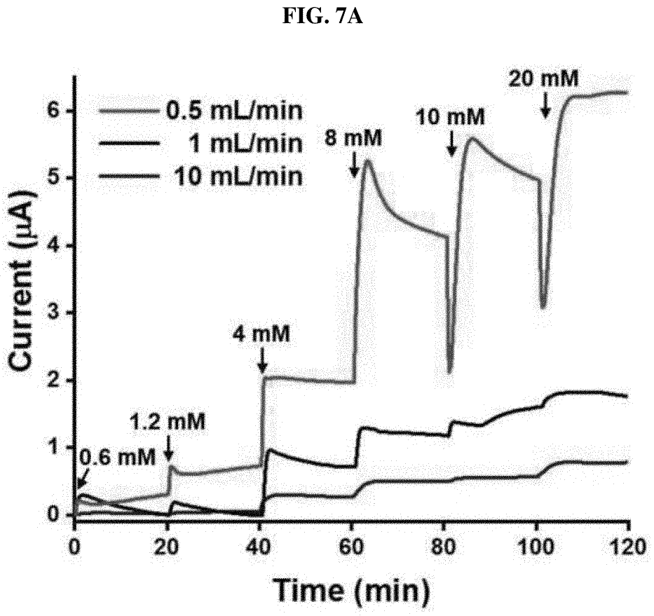

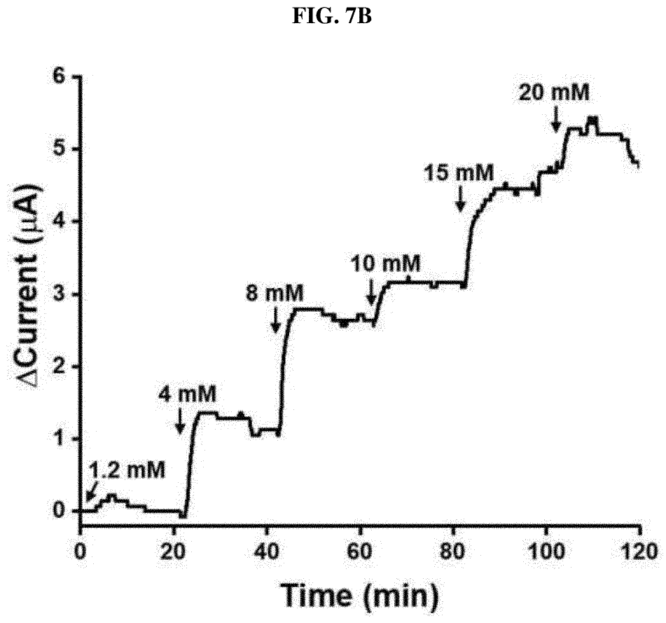

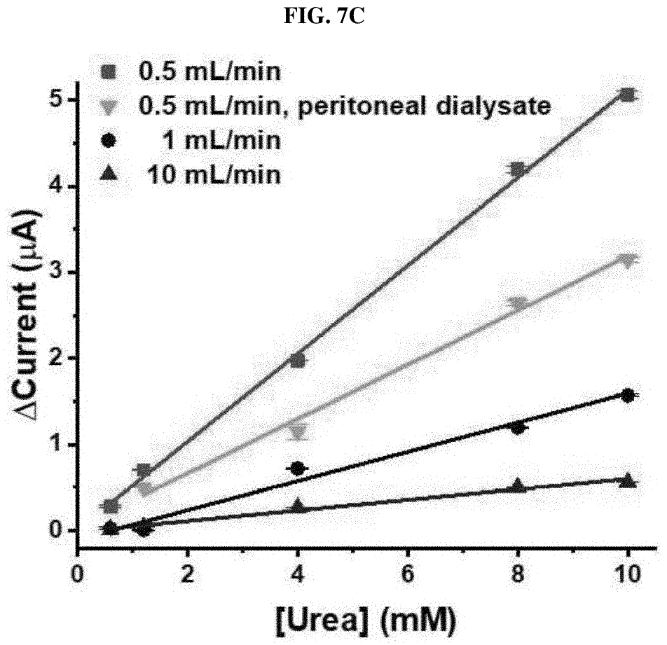

Optimized UAP membranes were assembled in a PDMS fluid chamber as shown in FIG. 1A. The manufactured urea sensor was tested using various numbers of the UAP membranes in the fluid chamber at a flow rate of 10 mL/min. The height of the chamber was 5.43 mm, and the maximum number of the membranes that can be inserted was 12. As shown in FIG. 6, four, six, eight, ten or twelve UAP membranes were inserted into the urea biosensor, and a urea sample was monitored using chronoamperometry at a constant voltage of 1.1V. In all cases, except the case using four membranes, a current increased according to the increase in urea concentration. This shows that urea monitoring under a flow condition can be implemented by chronoamperometry using six or more UAP membranes. When only four membranes were used (▴), a current decreased at a urea concentration of 8 mM or more. The height of the PDMS chamber was much higher than the total thickness of the four membranes. Therefore, the membrane may not be in contact with an electrode (system) due to the flow of the urea sample. In addition, in the configuration with four UAP membranes, a total of immobilized urease is less than in other configurations. Therefore, the decrease in current seems to be caused by the lack of immobilized urease and the long distance between the electrode and the membrane. The configuration of eight UAP membranes (▪) showed the highest current value for a given urea concentration. As the number of the membranes increased, the current increased, and the maximum value with respect to the configuration was measured. However, as the number of the membranes further increased, the current decreased. A current measured in the 12-membrane sensor (▾) was much lower than that in the 6-membrane sensor (●). It is considered that the decrease in current in a higher number of the UAP membranes is caused by the decrease in the flow of the urea sample adjacent to the electrode. When a higher number of the membranes were inserted, the membranes allowed the PDMS chamber to be overcharged. As a result, the urea sample did not permeate into the porous UAP membrane, but flowed with a fluid. This resulted in the decrease in a current measured by preventing the contact of the urea molecule with the immobilized urease. It was confirmed that the optimized number of the UAP membrane is 8. Accordingly, in a subsequent process, a urea sensor manufactured with eight UAP membranes was used to monitor urea. To perform real-time monitoring of a urea concentration under a flow condition, the urea sample was put into PBS, and allowed to flow into the PDMS chamber for 20 minutes at a flow rate of 0.5, 1 or 10 mL/min. Afterward, chronoamperometry was performed. The normal urea range in blood is 7 to 20 mg/dL (1.2 to 3.3 mM). Therefore, for measurement, the urea concentration was fixed in a range of 0.6 to 20 mM. The urea sensor of the present invention was manufactured to monitor urea concentrations in physiological samples, which are the same as blood and peritoneal fluid. Therefore, the maximum concentration of urea was set to 20 mM. The minimum concentration of urea was set to 0.6 mM which is approximately half of the minimum normal concentration. Therefore, the fixing of such a small value as the lowest value is advantageous for urea monitoring during dialysis. As shown in FIG. 7A, the maximum current in all ranges was measured at a flow rate of 0.5 mL/min (red), and a response time was calculated to less than 10 minutes. When the flow rates were 1 mL/min (black) and 10 mL/min (blue), currents measured with respect to low urea concentrations (0.6 and 1.2 mM) were not proportional. Anyway, the urea sensor based on the UAP membrane clearly showed concentration-dependent current values at other flow rates. To analyze an actual sample, after peritoneal fluid dialysis, the peritoneal fluid collected from a chronic renal failure patient was used. A urea concentration in the peritoneal fluid dialysate was calculated to 20 mM. Therefore, to prepare a monitoring sample, the dialysate was diluted with PBS. The samples were injected into the PDMS chamber at a flow rate of 0.5 mL/min and monitored using chronoamperometry. In the actual sample analysis, a minimum urea concentration of 1.2 mM was selected. As shown in FIG. 7B, the urea sensor based on the UAP membrane clearly showed a concentration-dependent current value with respect to a real sample. When the urea concentration was changed, the current value was immediately changed, and then became stable within 3 minutes, showing that the reaction time of the UAP-based urea sensor under a flow condition was three minutes. The fast response as described above enables efficient monitoring of a urea concentration in a physiological fluid in real time. Current values with respect to urea concentrations (0.6 to 10 mM) are shown in FIG. 7C. The results at all flow rates exhibited good linearity of R-square values (>0.99). At a flow rate of 0.5 mL/min, the limits of detection (LOD) in PBS (▪) and lung⋅peritoneal fluid dialysates (▾) were less than 0.6 mM and 1.2 mM, respectively. In addition, the LOD corresponding to the flow rates of 1 mL/min (●) and 10 mL/min (▴) were the same, that is, 4 mM. The sensitivities in PBS at the flow rates of 0.5, 1 and 10 mL/min were calculated to be 4.05, 1.31 and 0.46 mA·M⁻¹·cm⁻², respectively, and in the actual sample analysis, the sensitivity (▾) was calculated to be 2.4 mA·M⁻¹·cm⁻². However, the sensitivity was not significantly high, compared to that conventionally reported. Anyway, it is considered that this value is suitable for monitoring a urea concentration in a range of 0.6 to 20 mM. In addition, a method of measuring a urea concentration under a flow condition was not been reported yet. Therefore, the sensitivity and the LOD obtained in the present invention are considered to be significant. From the above-described data, applicability of the UAP membrane-based urea biosensor, which is operated with a proper sensitivity under a flow condition, was confirmed. It was confirmed that the biosensor of the present invention can be used in urea monitoring in a concentration range of 0.6 to 20 mM at a low flow rate of 0.5 mL/min. It was confirmed that, at a higher flow rate of more than 1 mL/min, the dynamic range of the urea biosensor was 4 to 20 mM. In addition, it was confirmed that the urea sensor manufactured in the present invention functions in lung⋅peritoneal fluid dialysates, which are physiological fluids, with suitable sensitivity and operation ranges under a flow condition. The urea biosensor of the present invention seems to be applicable to monitoring a urea concentration while maintaining a surgical operation, dialysis or an artificial kidney.

### CONCLUSIONS

In the present invention, to monitor a urea concentration under a flow condition, a portable urea sensor was manufactured based on a parylene-A-coated porous PTFE membrane. A 100-nm-thick parylene coating was applied on a porous PTFE membrane through vapor deposition. The parylene-A coating made the surface of the porous PTFE membrane functionalized with an amine group. It was confirmed that this coating is advantageous for the decrease in noise by decreasing non-specific binding, and facilitates the observation using a fluorescence microscope by functionalizing the membrane surface with an active amine group. Urease, which is a urea hydrolase, is immobilized to the AP membrane through glutaraldehyde crosslinking, thereby producing a specific electrochemical signal. The success of the urease immobilization process was confirmed by SEM and FTIR spectrometry. According to the above-described analyses, it was confirmed that the UAP membrane has a porous micro structure, and the urease is immobilized to the surface by a chemical bond. To manufacture a biosensor, the UAP membrane was inserted into a PDMS fluid chamber. A potential was applied using a screen-printed carbon electrode (system), and an electrochemical signal was detected. As results of urease activity assay and electrochemical measurement, it was found that, to produce the maximum signal, treatment with 48 mg/mL of urease was optimal. In addition, as a result of testing an effect with different numbers of UAP membranes, in the urea biosensor of the present invention, the configuration with eight UAP membranes exhibited the maximum current at a given urea concentration. Under an optimized condition, a urea sample was monitored while it flowed at different flow rates. The sensitivities at flow rates of 0.5, 1 and 10 mL/min were 4.05, 1.31 and 0.46 mA·M⁻¹·cm⁻², respectively. It was confirmed that the urea biosensor manufactured in the present invention was suitable for real-time monitoring of urea at flow rates ranging from 0.5 to 10 mL/min. In addition, as a result of testing the body fluid of a renal failure patient at a flow rate of 0.5 mL/min, sensitivity was calculated to be 2.4 mA·M⁻¹·cm⁻². As the urea sensor of the present invention preferably has a width of less than 4 cm, a length of less than 3 cm and a height of less than 2 cm, it is expected that is suitable for being applied to an artificial kidney, a portable dialysis system or the like.

The urea biosensor of the present invention can measure a urea concentration in a flowing sample with high sensitivity.

In addition, since the urea biosensor of the present invention can replace a urease-immobilized porous membrane as needed, a decrease in sensitivity due to the use for a long time can be prevented.

In addition, the urea biosensor of the present invention can reduce noise since urease is immobilized on the surface of a porous membrane by a chemical bond.

In addition, the portable urea biosensor of the present invention is suitable for being applied in an artificial kidney and a portable dialysis system.

In addition, in the urea biosensor of the present invention, urease is immobilized to a porous membrane by a chemical method, and since numerous urease-immobilized membranes are used as needed, an enzyme in the urea biosensor can be maintained at a high concentration, thus high sensitivity is increased.

In the paper Sensors 2018, 18, 2607, written by the inventor, Gun Yong, Sung, a urea sensor measuring flowing urea using silk fibroin as a urease-immobilized porous membrane is disclosed. This sensor can be operated only at a low flow rate, that is, 0.5 mL/min, and also can measure urea in a fluid sample at a high flow rate of 10 mL/min. In addition, in a previous paper disclosing the use of a silk fibroin membrane, while linearity is shown at a urea concentration of 0.3 to 1.2 mM, the present invention can measure urea even at a high concentration of 20 mM, showing a linear condition up to 10 mM, making it possible for a higher concentration of urea to be more accurately measured. Consequently, compared to the previous paper, the urea biosensor of the present invention can measure a higher concentration of urea with higher sensitivity under a higher flow rate of approximately 10 mL/min. It is determined that, compared to the previous paper and the prior patent, U.S. Pat. No. 1,871,781, an effect that cannot be easily predicted by one of ordinary skill in the art is exhibited.

It will be apparent to those skilled in the art that various modifications can be made to the above-described exemplary embodiments of the present invention without departing from the spirit or scope of the invention. Thus, it is intended that the present invention covers all such modifications provided they come within the scope of the appended claims and their equivalents.

What is claimed is:

1. A portable urea biosensor, comprising:
a fluid chamber consisting of a material that does not block an electrical signal;
parylene-A-coated insoluble porous membranes which are placed in the fluid chamber, and to which urease is immobilized by a chemical bond;
a screen-printed 3-electrode system, which is adjacent to the urease-immobilized parylene-A-coated insoluble porous membrane to which the urease is immobilized by a chemical bond, and includes a working electrode, a counter electrode and a reference electrode sensing an electrochemical signal generated from the membrane;
a sample inlet through which a flowing sample is introduced into the fluid chamber; and
a sample outlet through which the sample flows out from the fluid chamber,
wherein the urea biosensor measures a urea concentration by an electrochemical method with respect to a flowing sample.

2. The urea biosensor of claim 1, wherein the urease is immobilized to the urease-immobilized parylene-A-coated insoluble porous membrane by chemical crosslinking using glutaraldehyde.

3. The urea biosensor of claim 1, wherein the insoluble porous membrane is manufactured of one or more types of biocompatible materials selected from the group consisting of fucoidan, collagen, alginate, chitosan, hyaluronic acid, silk fibroin, a polyimide, polyamix acid, polycarprolactone, polyetherimide, nylon, polyaramid, polyvinyl alcohol, polyvinylpyrrolidone, poly-benzyl-glutamate, polyphenyleneterephthalamide, polyaniline, polyacrylonitrile, polyethylene oxide, polystyrene, cellulose, polyacrylate, polymethylmethacrylate, polylactic acid (PLA), polyglycolic acid (PGA), a copolymer (PLGA) of PLA and PGA, poly {poly(ethylene oxide)terephthalate-co-butyleneterephthalate} (PEOT/PBT), polyphosphoester (PPE), polyphosphazene (PPA), polyanhydride (PA), polytetrafluoroethylene (PTFE), poly(ortho ester) (POE), poly(propylene fumarate)-diacrylate (PPF-DA) and poly(ethylene glycol) diacrylate (PEG-DA).

4. The urea biosensor of claim 1, further comprising housings surrounding the fluid chamber and the 3-electrode system.

5. The urea biosensor of claim 1, wherein the working electrode of the 3-electrode system is aminated.

6. The urea biosensor of claim 1, wherein the 3-electrode system is connected with an external electrochemical analyzer to detect an electrochemical signal in the fluid chamber.

7. An insoluble porous membrane for immobilizing a protein on a surface of which is coated with an amine-functionalized parylene film.

8. The insoluble porous membrane of claim 7, wherein the insoluble porous membrane is manufactured of one or more biocompatible materials selected from the group consisting of fucoidan, collagen, alginate, chitosan, hyaluronic acid, silk fibroin, a polyimide, polyamix acid, polycarprolactone, polyetherimide, nylon, polyaramid, polyvinyl alcohol, polyvinylpyrrolidone, poly-benzyl-glutamate, polyphenyleneterephthalamide, polyaniline, polyacrylonitrile, polyethylene oxide, polystyrene, cellulose, polyacrylate, polymethylmethacrylate, polylactic acid (PLA), polyglycolic acid (PGA), a copolymer (PLGA) of PLA and PGA, poly{poly(ethylene oxide)terephthalate-co-butyleneterephthalate} (PEOT/PBT), polyphosphoester (PPE), polyphosphazene (PPA), polyanhydride (PA), polytetrafluoroethylene (PTFE), poly(ortho ester) (POE), poly(propylene fumarate)-diacrylate (PPF-DA) and poly(ethylene glycol) diacrylate (PEG-DA).

9. A method of manufacturing an insoluble porous membrane for immobilizing a protein, comprising:
(1) uniformly depositing an amine-functionalized parylene film on a porous membrane at room temperature; and
(2) after deposition of the parylene film, converting an amine group on the surface of the parylene film into an active aldehyde group by a reaction with a glutaraldehyde solution as a crosslinking agent.

10. The method of claim 9, wherein the step (1) is performed while a vacuum condition is maintained.

11. A method of manufacturing a urease-immobilized insoluble porous membrane, comprising:
(1) uniformly depositing an amine-functionalized parylene film on an insoluble porous membrane at room temperature;
(2) after deposition of the parylene film, converting an amine group on the surface of the parylene film into an active aldehyde group by a reaction with a glutaraldehyde solution as a crosslinking agent; and
(3) immobilizing urease to the insoluble porous membrane by a chemical reaction of the active aldehyde group on the surface of the parylene film and urease having a free amine group.

12. A urease-immobilized insoluble porous membrane for a urease biosensor, which is manufactured by the method of claim 11 and reduces noise in measurement of a urea concentration due to the decrease in non-specific reactions.

13. A method of measuring a urea concentration in a flowing sample by an electrochemical method using a portable urea biosensor which comprises a fluid chamber; parylene-A-coated insoluble porous membranes which are placed in the fluid chamber, and to which urease is immobilized by a chemical bond; a screen-printed 3-electrode system, which is adjacent to the insoluble porous membrane and includes a working electrode, a counter electrode and a reference electrode that sense an electrochemical signal generated from the insoluble porous membrane; a sample inlet through which a flowing sample is introduced into the fluid chamber; and a sample outlet through which the sample flows out from the fluid chamber.

14. The method of claim 13, wherein the flowing sample flows at a rate of 0.5 to 10 mL/min.

15. The method of claim 13, wherein the number of the urease-immobilized parylene-A-coated insoluble porous membranes is 6 to 10.

16. The method of claim 13, wherein the urea concentration in the sample ranges from 0.6 to 20 mM.
